(19) United States
(12) Patent Application Publication
(10) Pub. No.: US 2009/0105155 A1
(43) Pub. Date: Apr. 23, 2009

(54) TRUNCATED LHRH FORMULATIONS

(76) Inventors: David Charles Jackson, North Balwyn (AU); John Walker, Balwyn (AU); Weiguang Zeng, Kensington (AU)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/575,120

(22) PCT Filed: Sep. 12, 2005
(86) PCT No.: PCT/AU05/01383

§ 371 Date: Jan. 7, 2008

(30) Foreign Application Priority Data
Oct. 9, 2004 (AU) 2004905194

(51) Int. Cl. A61K 38/08; C07K 7/00; C07K 14/00

(52) U.S. Cl. 514/16; 530/329; 530/326; 530/325

(57) ABSTRACT

The present invention provides a peptide useful for raising an antiLHRH response in an animal. The peptide comprises a first and second region, the first region consisting of a sequence of less than 60 amino acids which comprises at least one T helper cell epitope and the second region consisting of the sequence SYGLRPG.

### FIELD OF INVENTION

This invention relates to peptides which comprise a truncated form of LHRH together with T helper epitopes. These formulations are of use in raising antibodies in animals directed against LHRH.

### BACKGROUND OF THE INVENTION

For any peptide to be able to induce an effective antibody response it must contain particular sequences of amino acids known as epitopes that are recognised by the immune system. In particular, for antibody responses, epitopes need to be recognised by specific immunoglobulin (Ig) receptors present on the surface of B lymphocytes. It is these cells which ultimately differentiate into plasma cells capable of producing antibody specific for that epitope. In addition to these B cell epitopes, the immunogen must also contain epitopes that are presented by antigen presenting cells (APC) to specific receptors present on helper T lymphocytes, the cells which are necessary to provide the signals required for the B cells to differentiate into antibody producing cells.

In the case of viral infections and in many cases of cancer, antibody is of limited benefit in recovery and the immune system responds with cytotoxic T cells (CTL) which are able to kill the virus-infected or cancer cell. Like helper T cells, CTL are first activated by interaction with APC bearing their specific peptide epitope presented on the surface, this time in association with MHC class I rather than class II molecules. Once activated the CTL can engage a target cell bearing the same peptide/class I complex and cause its lysis. It is also becoming apparent that helper T cells play a role in this process; before the APC is capable of activating the CTL it must first receive signals from the helper T cell to upregulate the expression of the necessary costimulatory molecules.

Helper T cell epitopes are bound by molecules present on the surface of APCs that are coded by class II genes of the major histocompatibility complex (MHC). The complex of the class II molecule and peptide epitope is then recognised by specific T-cell receptors (TCR) on the surface of T helper lymphocytes. In this way the T cell, presented with an antigenic epitope in the context of an MHC molecule, can be activated and provide the necessary signals for the B lymphocyte to differentiate. Traditionally the source of helper T cell epitopes for a peptide immunogen is a carrier protein to which peptides are covalently coupled but this coupling procedure can introduce other problems such as modification of the antigenic determinant during the coupling process and the induction of antibodies against the carrier at the expense of antibodies which are directed toward the peptide (Schutze, M. P., Leclerc, C. Jolivet, M. Audibert, F. Chedid, L. Carrier-induced epitopic suppression, a major issue for future synthetic vaccines. J Immunol. 1985, 135, 2319-2322; Dijohn, D., Torresi, J. R. Murillo, J. Herrington, D. A. et al. Effect of priming with carrier on response to conjugate vaccine. The Lancet. 1989, 2, 1415-1416). Furthermore, the use of irrelevant proteins in the preparation introduces issues of quality control. The choice of appropriate carrier proteins is very important in designing peptide vaccines and their selection is limited by factors such as toxicity and feasibility of their large scale production. There are other limitations to this approach including the size of the peptide load that can be coupled and the dose of carrier that can be safely administered (Audibert, F. a. C., L. 1984. Modern approaches to vaccines. Molecular and chemical basis of virus virulence and immunogenicity, Cold Spring Harbor Laboratory, New York). Although carrier molecules allow the induction of a strong immune response they are also associated with undesirable effects such as suppression of the anti-peptide antibody response (Herzenberg, L. A. and Tokuhisa, T. 1980. Carrier-priming leads to hapten-specific suppression. Nature 285: 664; Schutze, M. P., Leclerc, C., JOLIVET, M., Audibert, F., and Chedid, L. 1985. Carrier-induced epitopic suppression, a major issue for future synthetic vaccines. J Immunol 135: 2319; Etlinger, H. M., Felix, A. M., Gillessen, D., Heimer, E. P., JUST, M., Pink, J. R., Sinigaglia, F., Sturchler, D., Takacs, B., Trzeciak, A., 1988. Assessment in humans of a synthetic peptide-based vaccine against the sporozoite stage of the human malaria parasite, Plasmodium falciparum. J Immunol 140: 626).

In general then, an immunogen must contain epitopes capable of being recognised by helper T cells in addition to the epitopes that will be recognised by surface Ig or by the receptors present on cytotoxic T cells. It should be realised that these types of epitopes may be very different. For B cell epitopes, conformation is important as the B cell receptor binds directly to the native immunogen. In contrast, epitopes recognised by T cells are not dependent on conformational integrity of the epitope and consist of short sequences of approximately nine amino acids for CTL and slightly longer sequences, with less restriction on length, for helper T cells. The only requirements for these epitopes are that they can be accommodated in the binding cleft of the class I or class II molecule respectively and that the complex is then able to engage the T-cell receptor. The class II molecule's binding site is open at both ends allowing a much greater variation in the length of the peptides bound (Brown, J. H., T. S. Jardetzkyj J. C. Gorga, L. J. Stern, R. G. Urban, J. L. Strominger and D. C. Wiley. 1993. Three-dimensional structure of the human class II histocompatibility antigen HLA-DR1. Nature 364: 33) with epitopes as short as 8 amino acid residues being reported (Fahrer, A. M., Geysen, H. M., White, D. O., Jackson, D. C. and Brown, L. E. Analysis of the requirements for class II-restricted T-cell recognition of a single determinant reveals considerable diversity in the T-cell response and degeneracy of peptide binding to I-ED J. Immunol. 1995.155: 2849-2857).

LHRH (Luteinising hormone releasing hormone) is a ten amino acids long peptide hormone whose sequence is conserved in mammals. Its sequence is as follows:

pGlu-His-Trp-Ser-Tyr-Gly-Leu-Arg-Pro-Gly-NH
1 2 3 4 5 6 7 8 9 10

LHRH is secreted by the hypothalamus and controls the reproductive physiology of both males and females. The principle of development of LHRH-based immunocontraceptive vaccines is based on observations that antibodies to LHRH block the action of the hormone on pituitary secretion of luteinising hormone and follicle stimulating hormone, leading to gonadal atrophy and sterility in mammals.

Most LHRH vaccines that have been developed consist of LHRH chemically conjugated to protein carriers to provide T cell help for the generation of anti-LHRH antibodies. It has been shown that upon repeated inoculation of LHRH-protein carrier conjugates the anti-LHRH titre decreases due to the phenomenon known as “carrier induced epitope suppression”.

WO 88/05308 is an example of a disclosure of LHRH and fragments thereof linked to large protein carriers such as serum albumin and ovalbumin.

### SUMMARY OF THE INVENTION

The present inventors have found that a particular truncated form of LHRH, namely LHRH 4-10 (SYGLRPG), linked to a relatively small peptide including a T helper cell epitope(s) is particularly useful in the generation of an anti-LHRH response.

Accordingly in a first aspect the present invention provides a peptide comprising a first and second region, the first region consisting of a sequence of less than 60 amino acids which comprises at least one T helper cell epitope and the second region consisting of the sequence SYGLRPG.

In a second aspect the present invention provides a composition comprising a peptide comprising a first and second region, the first region consisting of a sequence of less than 60 amino acids which comprises at least one T helper cell epitope and the second region consisting of the sequence SYGLRPG and an acceptable carrier.

In a third aspect the present invention provides a lipopeptide, the lipopeptide comprising a first region, a second region and a third region, the first region consisting of a sequence of less than 60 amino acids which comprises at least one T helper cell epitope, the second region consisting of the sequence SYGLRPG and a third region comprising a lipid moiety wherein the second and third regions are covalently coupled to the first region.

In a fourth aspect the present invention provides a method of generating an anti LHRH response in an animal, the method comprising administering to the animal the peptide of the first aspect of the present invention, the composition of the second aspect of the present invention or the lipopeptide of the third aspect of the present invention.

In a fifth aspect the present invention provides the use of the peptide of the first aspect of the present invention or the lipopeptide of the third aspect of the present invention in the preparation of a medicament to induce an antiLHRH response in an animal.

### BRIEF DESCRIPTION OF FIGURES

FIG. 1 Anti-LHRH antibody titres in animals inoculated with LHRH (2-10) or LHRH (6-10)

FIG. 2 Testosterone levels in dogs inoculated with LHRH (2-10) or LHRH (6-10)

FIG. 3 Progesterone levels in dogs inoculated with LHRH (2-10) or LHRH (6-10)

FIG. 4 Anti-LHRH antibody titres induced by different priming and boosting regimes

FIG. 5 Testosterone levels Group 1: LHRH (6-10) prime, LHRH (6-10) boost

FIG. 6 Progesterone levels Group 1: LHRH (6-10) prime, LHRH (6-10) boost

FIG. 7 Testosterone levels Group 2: LHRH (2-10) prime, LHRH (2-10) boost

FIG. 8 Progesterone levels Group 2: LHRH (2-10) prime, LHRH (2-10) boost

FIG. 9 Testosterone levels Group 3: LHRH (2-10) prime, LHRH (6-10) boost

FIG. 10 Progesterone levels Group 3: LHRH (2-10) prime, LHRH (6-10) boost

FIG. 11 Anti-LHRH antibody titres induced by LHRH (6-10) & LHRH (4-10)

FIG. 12 Testosterone levels following vaccination with LHRH (6-10)

FIG. 13 Progesterone levels following vaccination with LHRH (6-10)

FIG. 14 Testosterone levels following vaccination with LHRH (4-10)

FIG. 15 Progesterone levels following vaccination with LHRH (4-10)

FIG. 16 Testes volume following vaccination with LHRH (4-10) and LHRH (6-10)

FIG. 17 Testes volume following vaccination with LHRH (4-10) and LHRH (6-10)

FIG. 18 Peptide sequences containing LHRH-related motifs from phage panning experiments.

### DETAILED DESCRIPTION OF THE INVENTION

The present inventors have found that a particular truncated form of LHRH, namely LHRH 4-10, linked to a relatively small peptide including a T helper cell epitope(s) is particularly useful in the generation of an anti-LHRH response.

Accordingly in a first aspect the present invention provides a peptide comprising a first and second region, the first region consisting of a sequence of less than 60 amino acids which comprises at least one T helper cell epitope and the second region consisting of the sequence SYGLRPG.

In a second aspect the present invention provides a composition comprising a peptide comprising a first and second region, the first region consisting of a sequence of less than 60 amino acids which comprises at least one T helper cell epitope and the second region consisting of the sequence SYGLRPG and an acceptable carrier.

In a third aspect the present invention provides a lipopeptide, the lipopeptide comprising a first region, a second region and a third region, the first region consisting of a sequence of less than 60 amino acids which comprises at least one T helper cell epitope, the second region consisting of the sequence SYGLRPG and a third region comprising a lipid moiety wherein the second and third regions are covalently coupled to the first region.

In a fourth aspect the present invention provides a method of generating an anti LHRH response in an animal, the method comprising administering to the animal the peptide of the first aspect of the present invention, the composition of the second aspect of the present invention or the lipopeptide of the third aspect of the present invention.

In a fifth aspect the present invention provides the use of the peptide of the first aspect of the present invention or the lipopeptide of the third aspect of the present invention in the preparation of a medicament to induce an antiLHRH response in an animal.

In a preferred embodiment the C-terminal of the first region is consists of less than 40, preferably less than 20, amino acids.

In a further preferred embodiment the first region comprises 1, 2 or 3 T helper cell epitopes, preferably one T helper cell epitope.

In another preferred embodiment the C-terminal residue of the first region is linked to the N-terminal reside of the second region.

In a further preferred embodiment each T helper cell epitope is selected from the group consisting of SSKTQTHTQQDRPPQPS; QPSTELEETRTSRARHS; RHSTTSAQRSTHYDPRT; PRTSDRPVSYTMNRTRS; TRSRKQTSHRLKNIPVH; SHQYLVIKLIPNASLIE; IGTDNVHYKIMTRPSHQ; YKIMTRPSHQYLVIKLI; KLIPNASLIENCTKAEL; AELGEYEKLLNSVLEPI; KLLNSVLEPINQALTLM; EPINQALTLMTKNVKPL; FAGWLAGVALGVATAA; GVALGVATAAQITAGIA; TAAQITAGIALHQSNLN; GIALHQSNLNAQAIQSL; NLNAQAIQSLRTSLEQS; QSLRTSLEQSNKAIEEI; EQSNKAIEEIREATQET; TELLSIFGPSLRDPISA; PRYIATNGYLISNFDES; CIRGDTSSCARTLVSGT; DESSCVFVSESAICSQN; TSTIINQSPDKLLTFIA, SPDKLLTFIASDTCPLV, SGRRQRRFAGWLAGVA and combinations thereof.

In a further preferred embodiment the peptide has a sequence selected from the group consisting of QPSTELEETRTSRARHSSYGLRPG, TRSRKQTSHRLKNIPVHSYGLRPG, SHQYLVIKLIPNASLIESYGLRPG, KLIPNASLIENCTKAELSYGLRPG, AELGEYEKLLNSVLEPISYGLRPG, TAAQITAGIALHQSNLNSYGLRPG and PRYIATNGYLISNFDESSYGLRPG.

Further information regarding T helper cell epitopes which may be used in the present invention is provided in WO 00/46390, the disclosure of which is incorporated herein by cross reference.

As mentioned above the composition of the second aspect of the present invention comprises an acceptable carrier. It is preferred that the carrier is an adjuvant.

Acceptable carriers or diluents include those used in compositions suitable for oral, rectal, nasal, topical (including buccal and sublingual), vaginal, parenteral (including subcutaneous, intramuscular, intravenous, intradermal, intrathecal and epidural) administration. They are non-toxic to recipients at the dosages and concentrations employed.

Representative examples of pharmaceutically acceptable carriers or diluents include, but are not limited to water, isotonic solutions which are preferably buffered at a physiological pH (such as phosphate buffered saline or Tris-buffered saline) and can also contain one or more of, mannitol, lactose, trehalose, dextrose, glycerol, ethanol or polypeptides (such as human serum albumin). The compositions may conveniently be presented in unit dosage form and may be prepared by any of the methods well known in the art of pharmacy.

As mentioned it is preferred that the composition includes an adjuvant. As will be understood an “adjuvant” means a composition comprised of one or more substances that enhances the immunogenicity and efficacy of a vaccine composition. Non-limiting examples of suitable adjuvants include squalane and squalene (or other oils of animal origin); block copolymers; detergents such as Tween®-80; QUIL® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® adjuvant; mycobacterial cell wall extract; synthetic glycopeptides such as MURAMYL dipeptides or other derivatives; Avridine; Lipid A derivatives; dextran sulfate; DEAE-DEXTRAN or with aluminium phosphate; carboxypolymethylene such as Carbopol'EMA; acrylic copolymer emulsions such as Neocryl A640 (e.g. U.S. Pat. No. 5,047,238); vaccinia or animal poxvirus proteins; sub-viral particle adjuvants such as cholera toxin, or mixtures thereof.

The peptides of the present invention may be produced in a number of ways, however, it is preferred that the peptides are produced synthetically using methods well known in the field. For example, the peptides may be synthesised using solution synthesis or solid phase synthesis as described, for example, in Chapter 9 entitled “Peptide Synthesis” by Atherton and Sheppard which is included in a publication entitled “Synthetic Vaccines” edited by Nicholson and published by Blackwell Scientific Publications. Preferably a solid phase support is utilised which may be polystyrene gel beads wherein the polystyrene may be cross-linked with a small proportion of divinylbenzene (e.g. 1%) which is further swollen by lipophilic solvents such as dichloromethane or more polar solvents such as dimethylformamide (DMF). The polystyrene may be functionalised with chloromethyl or aminomethyl groups. Alternatively, cross-linked and functionalised polydimethyl-acrylamide gel is used which may be highly solvated and swollen by DMF and other dipolar aprotic solvents. Other supports can be utilised based on polyethylene glycol which is usually grafted or otherwise attached to the surface of inert polystyrene beads. In a preferred form, use may be made of commercial solid supports or resins which are selected from PAL-PEG-PS, PAC-PEG-PS, KA, KR or TGR.

In solid state synthesis, use is made of reversible blocking groups which have the dual function of masking unwanted reactivity in the α-amino, carboxy or side chain functional groups and of destroying the dipolar character of amino acids and peptides which render them inactive. Such functional groups can be selected from t-butyl esters of the structure RCO—OCMe. Use may also be made of the corresponding benzyl esters having the structure RCO—OCH₂—C₆H₅ and urethanes having the structure C₆H₅CH₂OCO—NHR which are known as the benzyloxycarbonyl or Z-derivatives and any Me₃-COCO—NHR, which are known as t-butoxyl carbonyl, or Boc derivatives. Use may also be made of derivatives of fluorenyl methanol and especially the fluorenyl-methoxy carbonyl or Fmoc group. Each of these types of protecting group is capable of independent cleavage in the presence of one other so that frequent use is made, for example, of BOC-benzyl and Fmoc-tertiary butyl protection strategies.

Reference also should be made to a condensing agent to link the amino and carboxy groups of protected amino acids or peptides. This may be done by activating the carboxy group so that it reacts spontaneously with a free primary or secondary amine. Activated esters such as those derived from p-nitrophenol and pentafluorophenol may be used for this purpose. Their reactivity may be increased by addition of catalysts such as 1-hydroxybenzotriazole. Esters of triazine DHBT (as discussed on page 215-216 of the abovementioned Nicholson reference) also may be used. Other acylating species are formed in situ by treatment of the carboxylic acid (i.e. the N-alpha-protected amino acid or peptide) with a condensing reagent and are reacted immediately with the amino component (the carboxy or C-protected amino acid or peptide). Dicyclohexylcarbodiimide, the BOP reagent (referred to on page 216 of the Nicholson reference), O'Benzotriazole-N,N,N′N′-tetra methyl-uronium hexafluorophosphate (HBTU) and its analogous tetrafluoroborate are frequently used condensing agents.

The attachment of the first amino acid to the solid phase support may be carried out using BOC-amino acids in any suitable manner. In one method BOC amino acids are attached to chloromethyl resin by warming the triethyl ammonium salts with the resin. Fmoc-amino acids may be coupled to the p-alkoxybenzyl alcohol resin in similar manner. Alternatively, use may be made of various linkage agents or “handles” to join the first amino acid to the resin. In this regard, p-hydroxymethyl phenylacetic acid linked to aminomethyl polystyrene may be used for this purpose.

As mentioned above in a third aspect the present invention provides a lipopeptide. The lipopeptide is preferably a “branched” structure and additional details regarding such lipopeptides may be found in WO 04/014956 and WO 04/014957, the disclosures of which are incorporated by cross reference. Alternatively the lipid moiety may be simply attached to the N-terminal of the peptide.

Accordingly, in a third aspect the present invention provides a lipopeptide, the lipopeptide comprising a first region, a second region and a third region, the first region consisting of a sequence of less than 60 amino acids which comprises at least one T helper cell epitope, the second region consisting of the sequence SYGLRPG and a third region comprising a lipid moiety wherein the second and third regions are covalently coupled to the first region.

In a preferred embodiment the first region consists of less than 40, preferably less than 20, amino acids. It is further preferred that the first region comprises 1, 2 or 3 T helper cell epitopes, preferably one T helper cell epitope.

In a further preferred embodiment the lipid moiety is coupled to the C-terminal of the first region. In addition, it is preferred that the C-terminal residue of the first region is linked to the N-terminal residue of the second region.

In another preferred embodiment the C-terminal residue of the first region is lysine or an analogue thereof and the lipid moiety is linked to the ε-amino group and the second region is linked to the carboxyl group. Alternatively, the lysine or analogue thereof is linked to the remainder of the first region via its ε-amino group, the lipid moiety is linked to the α-amino group, and the second region is linked to the carboxyl group.

Examples of lysine analogues include ornithine, diaminoproprionic acid, and diaminobutyric acid.

In yet another preferred embodiment the T helper cell epitope is selected from the group consisting of SSKTQTHTQQDRPPQPS; QPSTELEETRTSRARHS; RHSTTSAQRSTHYDPRT; PRTSDRPVSYTMNRTRS; TRSRKQTSHRLKNIPVH; SHQYLVIKLIPNASLIE; IGTDNVHYKIMTRPSHQ; YKIMTRPSHQYLVIKLI; KLIPNASLIENCTKAEL; AELGEYEKLLNSVLEPI; KLLNSVLEPINQALTLM; EPINQALTLMTKNVKPL; FAGWLAGVALGVATAA; GVALGVATAAQITAGIA; TAAQITAGIALHQSNLN; GIALHQSNLNAQAIQSL; NLNAQAIQSLRTSLEQS; QSLRTSLEQSNKAIEEI; EQSNKAIEEIREATQET; TELLSIFGPSLRDPISA; PRYIATNGYLISNFDES; CIRGDTSSCARTLVSGT; DESSCVFVSESAICSQN; TSTIINQSPDKLLTFIA, SPDKLLTFIASDTCPLV, SGRRQRRFAGWLAGVA and combinations thereof.

It is also preferred that the sequence of the first region of the lipopeptide is selected from the group consisting of QPSTELEETRTSRARHSK, TRSRKQTSHRLKNIPVHK, SHQYLVIKLIPNASLIEK, KLIPNASLIENCTKAELK, AELGEYEKLLNSVLEPIK, TAAQITAGIALHQSNLNK and PRYIATNGYLISNFDESK.

It is preferred that the lipid moiety is a lipoamino acid moiety and is preferably selected from the group consisting of Pam₂Cys, Pam₃Cys, Ste₂Cys, Lau₂Cys, Oct₂Cys, Pam₂Asp, Pam₃Asp, Ste₂Asp, Lau₂Asp, and Oct₂Asp, and is most preferably Pam₂Cys or Pam₃Cys.

As will be known to those skilled in the art Pam₂Cys is also known as N-palmitoyl-S-[2,3-bis(palmitoyloxy)propyl]cysteine, Pam₂Cys is also known as dipalmitoyl-S-glyceryl-cysteine or S-[2,3-bis(palmitoyloxy)propyl]cysteine, Ste₂Cys is also known as S-[2,3-bis(stearoyloxy)propyl]cysteine or distearoyl-S-glyceryl-cysteine; that Lau₂Cys is also known as S-[2,3-bis(lauroyloxy)propyl]cysteine or dilauroyl-S-glyceryl-cysteine); and that Oct₂Cys is also known as S-[2,3-bis(octanoyloxy)propyl]cysteine or dioctanoyl-S-glyceryl-cysteine).

In a preferred embodiment the lipid moiety is linked to the first region via a spacer. Preferably the spacer comprises an arginine or serine dimers, trimers or teramers, etc. Alternatively, a 6-aminohexanoic acid spacer can be used.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In order that the nature of the present invention may be more clearly understood preferred forms thereof will now be described with reference to the following Examples.

### EXAMPLES

### Peptides

Peptides incorporating various fragments of LHRH and T helper cell epitopes were synthesised using standard techniques.

Each of the peptides included one of the T helper cell epitopes disclosed in WO 00/46390. The sequences of these T helper cell epitopes were as follows:

| | |
|---|---|
| P4 | QPSTELEETRTSRARHS |
| P10 | TRSRKQTSHRLKNIPVH |
| P24 | SHQYLVIKLIPNASLIE |
| P25 | KLIPNASLIENCTKAEL |
| P27 | AELGEYEKLLNSVLEPI |
| P35 | TAAQITAGIALHQSNLN |
| P62 | PRYIATNGYLISNFDES |

### Example 1

Groups of dogs received 5 nmoles total peptide consisting of a pool of peptides P25, P35 and P62 coupled to LHRH (2-10) as a first dose. For second and third doses one group of dogs received the same vaccine whereas a second group received vaccine consisting of P25 only coupled to LHRH (6-10). The dogs received the three doses of vaccine in week 0, 4 and 14.

Preparation of the vaccine: 1 to 1.5 mg of each of the peptides were weighed out and dissolved in 100 ul of 4 M urea separately. The amounts of the solutions corresponding to 1.7 nmoles of each peptide were mixed and diluted with isotonic saline to the designated dose volume for injection (1 ml per dose). For each dose 150 ug of Iscomatrix•® (CSL, Melbourne, Australia) was also added as adjuvant. The vaccine was given to dogs in the scruff of the neck.

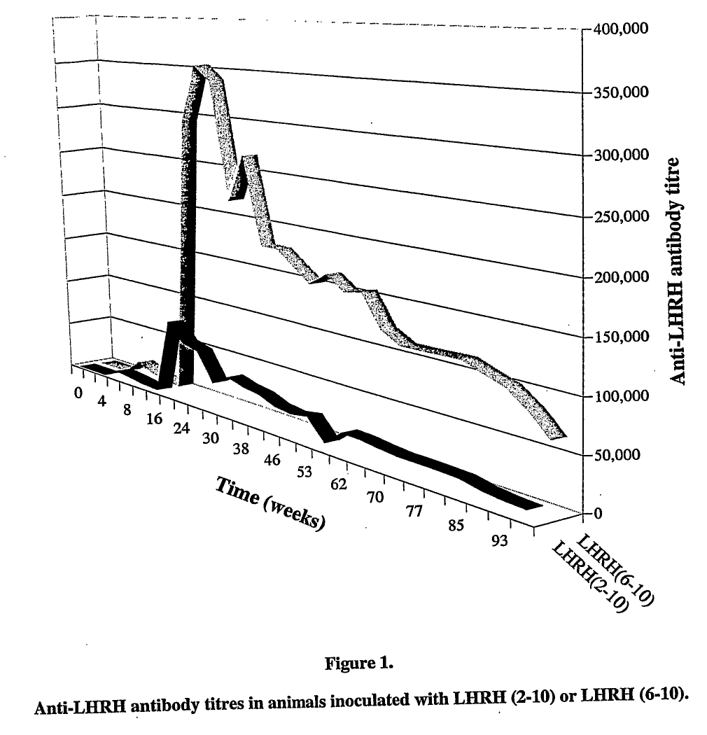

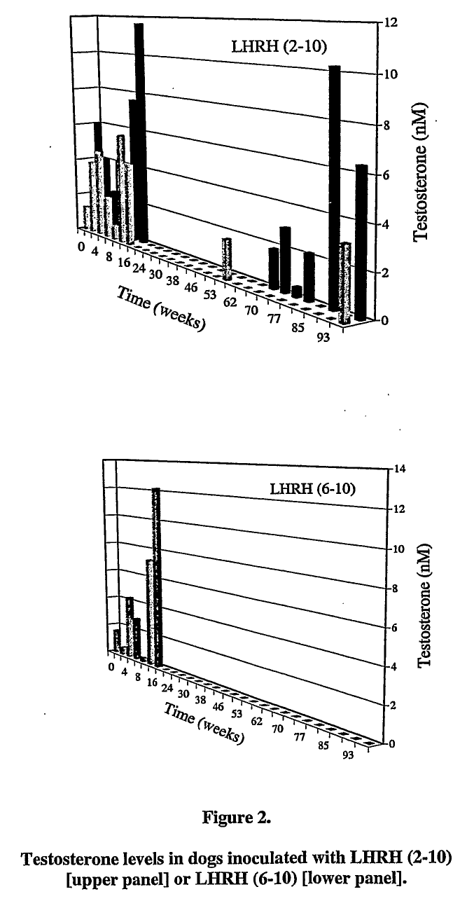

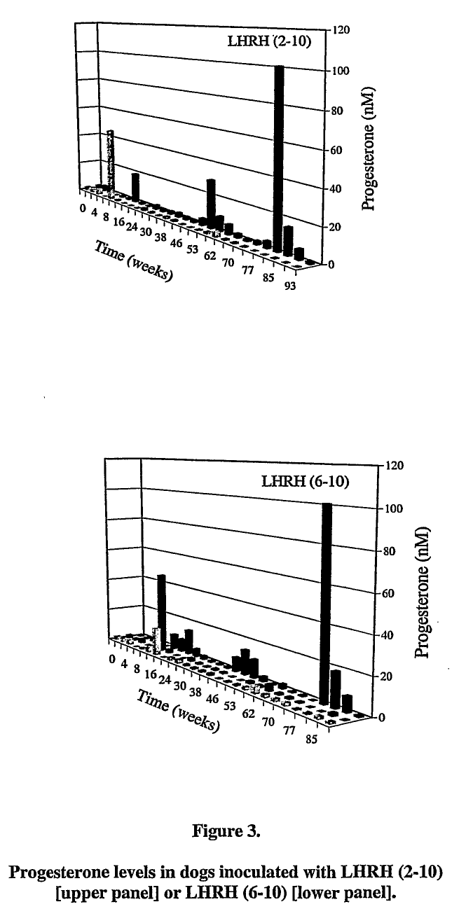

The antibody titres obtained are shown in FIG. 1. whilst testosterone and progesterone levels are shown in FIGS. 2 and 3 respectively.

### Example 2

### Inoculation Regimes

Beagles/foxhound dogs were divided into three groups. One group (12 dogs) received three doses of 5 nmoles of total peptides consisting of a pool of peptides p25, p35 and p62 coupled to LHRH (6-10).

A second group (6 dogs) received three doses of 5 nmoles of total peptides consisting of a pool of peptides p25, p35 and p62 coupled to LHRH (2-10).

A third group (6 dogs) received three doses of 5 nmoles of total peptides consisting of a pool of peptides p25, p35 and p62 coupled to LHRH (2-10) as the first dose. For the second and third dose the vaccine was the same pool of peptides but coupled to LHRH (6-10).

The dogs received the three doses of vaccine in week 0, 4 and 14 respectively.

### Preparation of the Vaccine.

1 to 1.5 mg of each of the peptides were weighed out and dissolved in 100 ul of 4 M urea separately. The amounts of the solutions corresponding to 1.7 nmoles of each peptide were mixed and diluted with isotonic saline to the designated dose volume for injection (1 ml per dose). For each dose 150 ug of Iscomatrix•® (CSL, Melbourne, Australia) was also added as adjuvant. The vaccine was given to dogs in the scruff of the neck.

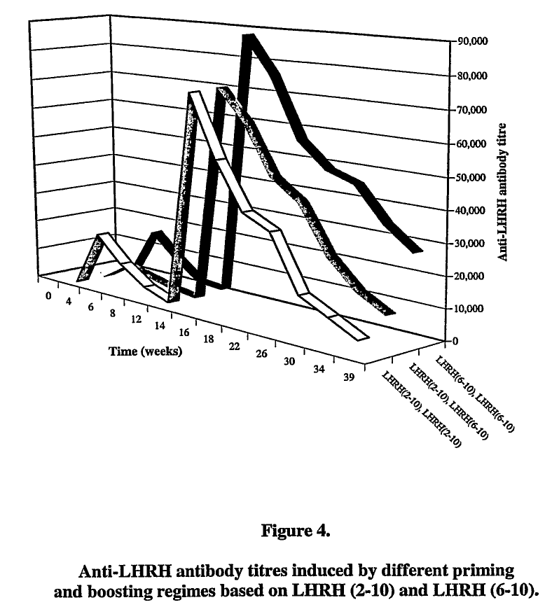

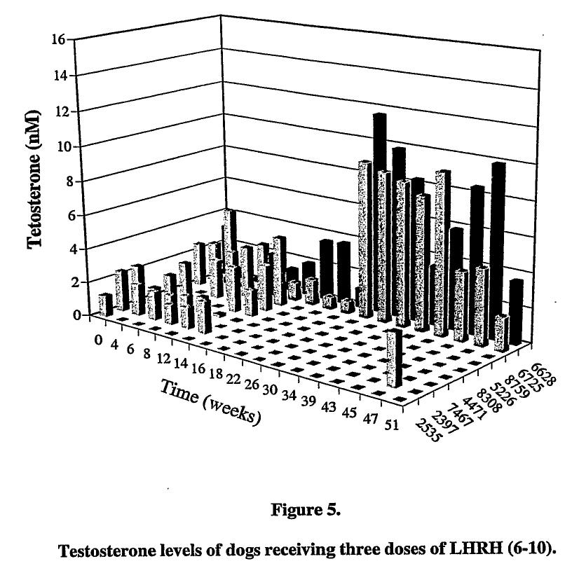

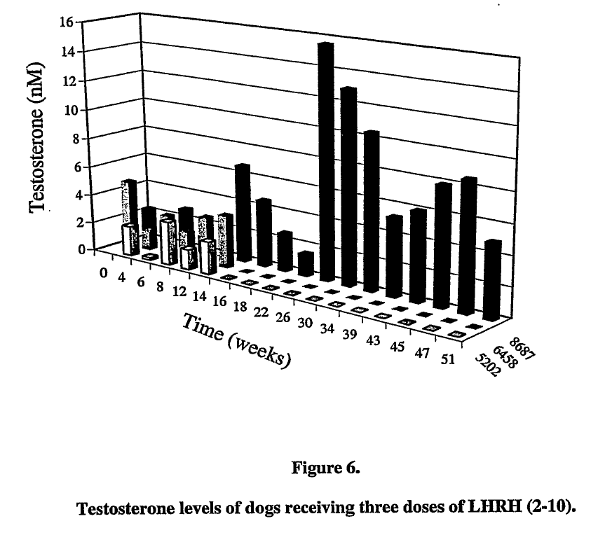

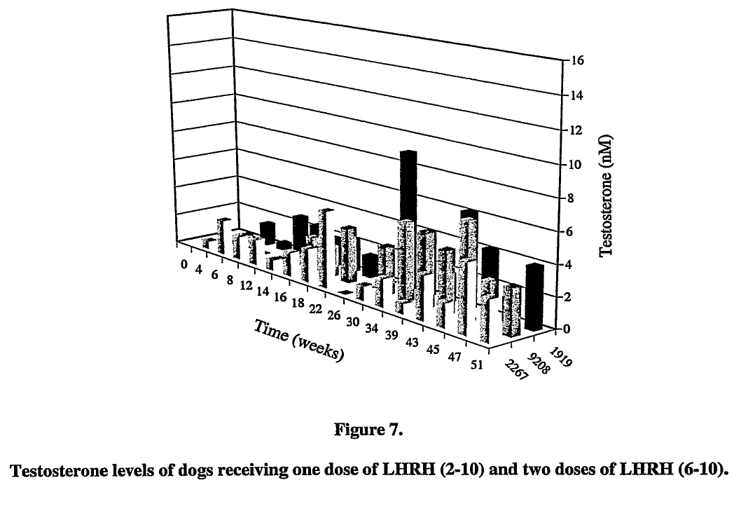

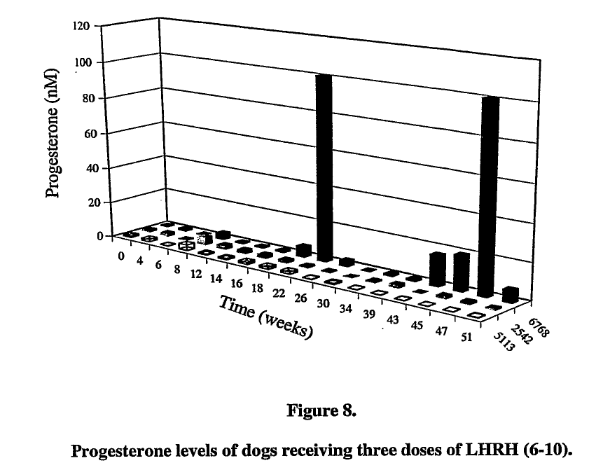

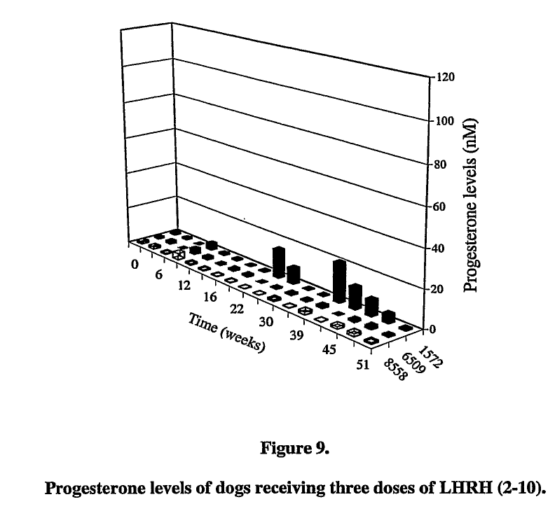

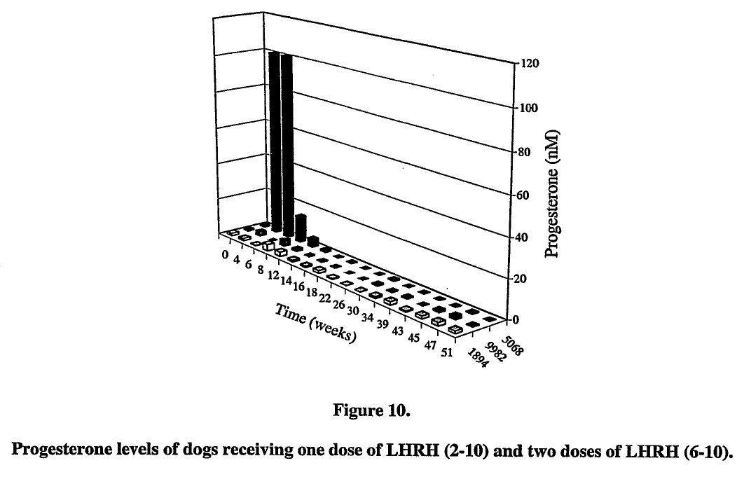

The results of these experiments are shown in FIGS. 4 to 10.

There is little apparent difference here in the titres of antibodies following the second dose of vaccine whether elicited by LHRH (2-10) or LHRH (6-10). The regime of vaccination also seemed to make no difference i.e. whether they were administered in the order: LHRH (2-10) [primary dose] followed by LHRH (2-10) [boost]; LHRH (2-10) [primary] followed by LHRH (6-10) [boost] or LHRH (6-10) [primary] followed by LHRH (6-10) [boost]. There is, however, a difference in titre in the primary antibody response with LHRH (2-10)-LHRH (6-10) showing the lowest titres. In regard to testosterone and progesterone some differences can be seen with apparently a larger proportion of animals vaccinated with two doses of LHRH (6-10) displaying lower levels of testosterone and progesterone.

### Example 3

### Inoculation Regimes

Beagles/foxhound dogs were divided into two groups. One group (15 dogs) received three doses of 35 nmoles of total peptides consisting of a pool of peptides p4, p10, p24, p25, p27, p35 and p62 coupled to LHRH (6-10).

A second group (15 dogs) received three doses of 35 nmoles of total peptides consisting of a pool of peptides p4, p10, p24, p25, p27, p35 and p62 coupled to LHRH (4-10).

The dogs received the three doses of vaccine in week 0, 4 and 14 respectively.

### Preparation of the Vaccine:

1 to 1.5 mg of each of the peptides were weighed out and dissolved in 100 ul of 4 M urea separately. The amounts of the solutions corresponding to 5 nmoles of each peptide were mixed and diluted with isotonic saline to the designated dose volume for injection (1 ml per dose). For each dose 150 ug of Iscomatrix•® (CSL, Melbourne, Australia) was also added as adjuvant. The vaccine was given to dogs in the scruff of the neck.

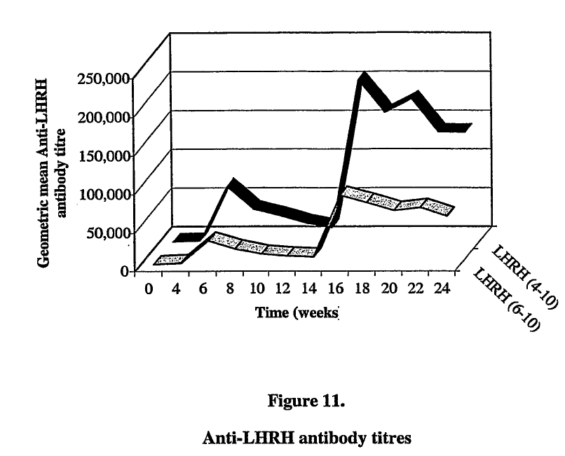

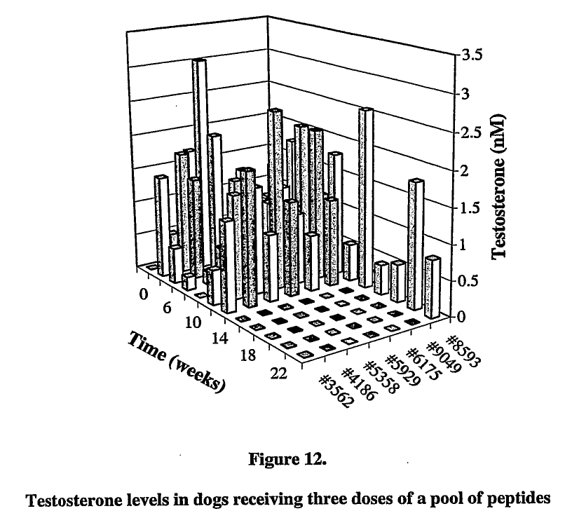

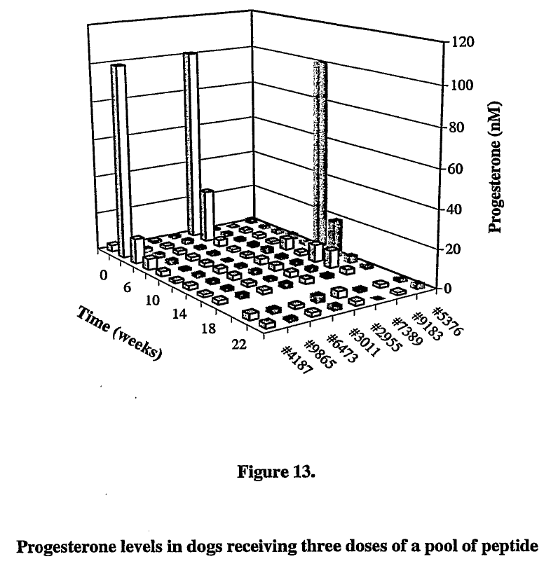

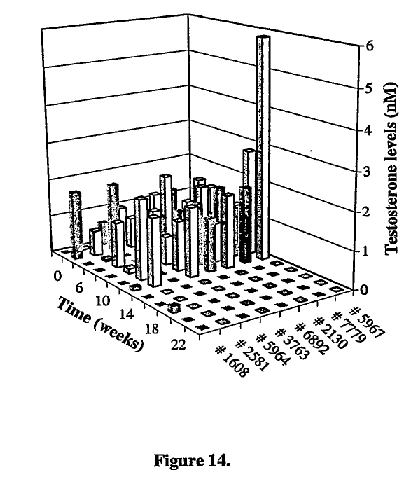

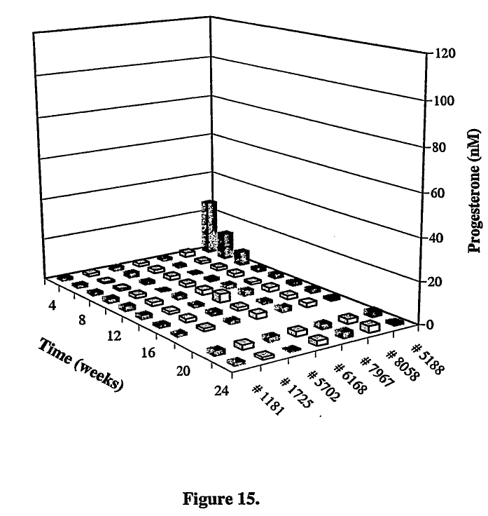

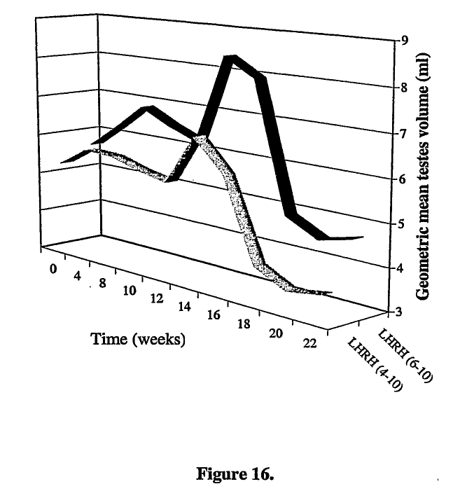

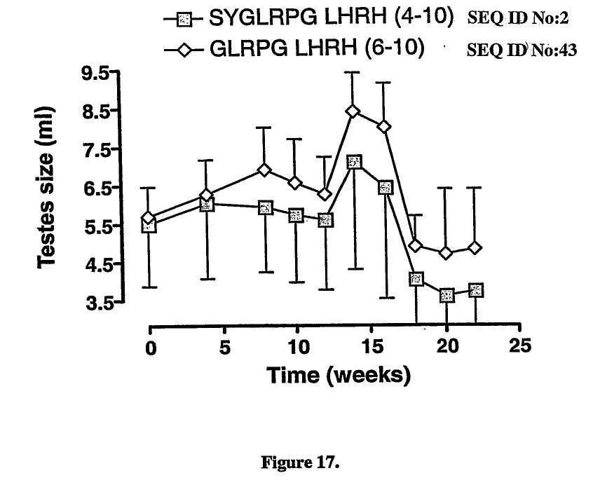

The results of these experiments are shown in FIGS. 11 to 17.

Higher titres of antibodies were obtained in animals vaccinated with LHRH (4-10). Further it was LHRH (4-10) appeared to induce antibodies which have a more striking effect on testosterone and progesterone levels; unlike animals inoculated with LHRH (6-10), no animals show hormone breakthrough when inoculated with LHRH (4-10).

### Example 4

### Peptide Sequences Containing LHRH-Related Motifs from Phage Panning Experiments

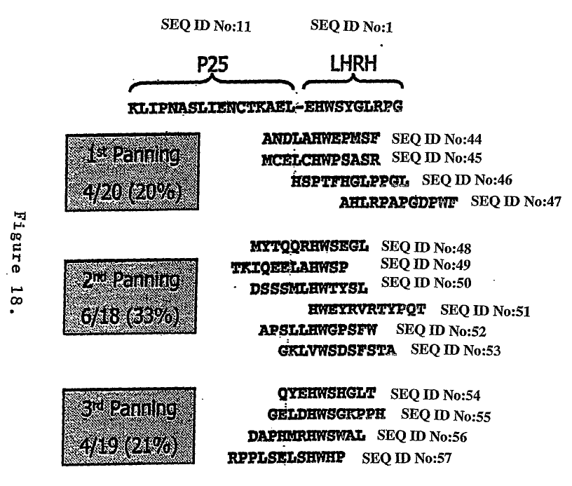

The peptide sequences shown in FIG. 18 were obtained from panning using anti-P25-LHRH (2-10) antibodies and acid elution. A total of twenty phage clones were randomly picked for sequence analysis after each of the three pannings without ELISA screening. Amino acid residues which are identical to the P25-LHRH (2-10) sequences were highlighted. The total number related sequence motifs out of the total number of successfully PCR/sequenced clones are given in the shaded boxes followed by the percentage in parenthesis. In this way “4/20 (20%)” indicates that the LHRH-related motifs were present in 4 of the 20 successfully sequenced clones which represents 20% of the clones analysed after first panning.

From these results it appears that the use of a larger LHRH fragment biases the antibody response to the C-terminal of the sequence.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A peptide comprising a first and second region, the first region consisting of a sequence of less than 60 amino acids which comprises at least one T helper cell epitope and the second region consisting of the sequence SYGLRPG SEQ ID No:2.

2. The peptide according to claim 1 wherein the first region consists of less than 40 amino acids.

3. The peptide according to claim 1 wherein the first region comprises 1, 2 or 3 T helper cell epitopes.

4. The peptide according to claim 1 wherein the C-terminal residue of the first region is linked to the N-terminal residue of the second region.

5. The peptide according to claim 1 wherein the T helper cell epitope is selected from the group consisting of SSKTQTHTQQDRPPQPS SEQ ID No:3; QPSTELEETRTSRARHS SEQ ID No:4; RHSTTSAQRSTHYDPRT SEQ ID No:5; PRTSDRPVSYTMNRTRS SEQ ID No:6; TRSRKQTSHRLKNIPVH SEQ ID No:7; SHQYLVIKLIPNASLIE SEQ ID No:8; IGTDNVHYKIMTRPSHQ SEQ ID No:9; YKIMTRPSHQYLVIKLI SEQ ID No:10; KLIPNASLIENCTKAEL SEQ ID No:11; AELGEYEKLLNSVLEPI SEQ ID No:12; KLLNSVLEPINQALTLM SEQ ID No:13; EPINQALTLMTKNVKPL SEQ ID No:14; FAGWLAGVALGVATAA SEQ ID No:15; GVALGVATAAQITAGIA SEQ ID No:16; TAAQITAGIALHQSNLN SEQ ID No:17; GIALHQSNLNAQAIQSL SEQ ID No:18; NLNAQAIQSLRTSLEQS SEQ ID No:19; QSLRTSLEQSNKAIEEI SEQ ID No:20; EQSNKAIEEIREATQET SEQ ID No:21; TELLSIFGPSLRDPISA SEQ ID No:22; PRYIATNGYLISNFDES SEQ ID No:23; CIRGDTSSCARTLVSGT SEQ ID No:24; DESSCVFVSESAICSQN SEQ ID No:25; TSTIINQSPDKLLTFIA SEQ ID No:26, SPDKLLTFIASDTCPLV SEQ ID No:27, SGRRQRRFAGWLAGVA SEQ ID No:28 and combinations thereof.

6. The peptide according to claim 1 wherein the peptide has a sequence selected from the group consisting of

| | |
|---|---|
| QPSTELEETRTSRARHSSYGLRPG, | SEQ ID No:29 |
| TRSRKQTSHRLKNIPVHSYGLRPG, | SEQ ID No:30 |
| SHQYLVIKLIPNASLIESYGLRPG, | SEQ ID No:31 |
| KLIPNASLIENCTKAELSYGLRPG, | SEQ ID No:32 |
| AELGEYEKLLNSVLEPISYGLRPG, | SEQ ID No:33 |
| TAAQITAGIALHQSNLNSYGLRPG | SEQ ID No:34 |
| and | |
| PRYIATNGYLLSNFDESSYGLRPG. | SEQ ID No:35 |

7. A composition comprising the peptide of claim 1 and an acceptable carrier.

8. The composition according to claim 7 wherein the carrier is an adjuvant.

9. A lipopeptide, the lipopeptide comprising a first region, a second region and a third region, the first region consisting of a sequence of less than 60 amino acids which comprises at least one T helper cell epitope, the second region consisting of the sequence SYGLRPG SEQ ID No:2 and a third region comprising a lipid moiety wherein the second and third regions are covalently coupled to the first region.

10. The lipopeptide according to claim 9 wherein the first region consists of less than 40 amino acids.

11. The lipopeptide according to claim 9 wherein the first region comprises 1, 2 or 3 T helper cell epitopes.

12. The lipopeptide according to claim 9 wherein the C-terminal residue of the first region is linked to the N-terminal residue of the second region.

13. The lipopeptide according to claim 9 wherein the lipid moiety is coupled to the C-terminal residue of the first region.

14. The lipopeptide according to claim 9 wherein the C-terminal residue of the first region is lysine or an analogue thereof and the lipid moiety is linked to the ε-amino group and the second region is linked to the carboxyl group.

15. The lipopeptide according to claim 9 wherein the C-terminal residue of the first region is lysine or an analogue thereof and the lysine or analogue thereof is linked to the remainder of the first region via its ε-amino group, the lipid moiety is linked to the α-amino group, and the second region is linked to the carboxyl group.

16. The lipopeptide according to claim 14 wherein the lysine analogue is ornithine, diaminoproprionic acid, or diaminobutyric acid.

17. The lipopeptide according to claim 9 wherein the lipid moiety is coupled to the N-terminal residue of the first region.

18. The lipopeptide according to claim 9 wherein the T helper cell epitope is selected from the group consisting of SSKTQTHTQQDRPPQPS SEQ ID No:3; QPSTELEETRTSRARHS SEQ ID No:4; RHSTTSAQRSTHYDPRT SEQ ID No:5; PRTSDRPVSYTMNRTRS SEQ ID No:6; TRSRKQTSHRLKNIPVH SEQ ID No:7; SHQYLVIKLIPNASLIE SEQ ID No:8; IGTDNVHYKIMTRPSHQ SEQ ID No:9; YKIMTRPSHQYLVIKLI SEQ ID No:10; KLIPNASLIENCTKAEL SEQ ID No:11; AELGEYEKLLNSVLEPI SEQ ID No:12; KLLNSVLEPINQALTLM SEQ ID No:13; EPINQALTLMTKNVKPL SEQ ID No:14; FAGWLAGVALGVATAA SEQ ID No:15; GVALGVATAAQITAGIA SEQ ID No:16; TAAQITAGIALHQSNLN SEQ ID No:17; GIALHQSNLNAQAIQSL SEQ ID No:18; NLNAQAIQSLRTSLEQS SEQ ID No:19; QSLRTSLEQSNKAIEEI SEQ ID No:20; EQSNKAIEEIREATQET SEQ ID No:21; TELLSIFGPSLRDPISA SEQ ID No:22; PRYIATNGYLISNFDES SEQ ID No:23; CIRGDTSSCARTLVSGT SEQ ID No:24; DESSCVFVSESAICSQN SEQ ID No:25; TSTIINQSPDKLLTFIA SEQ ID No:26, SPDKLLTFIASDTCPLV SEQ ID No:27, SGRRQRRFAGWLAGVA SEQ ID No:28 and combinations thereof.

19. The lipopeptide according to claim 9 wherein the sequence of the first region is selected from the group consisting of QPSTELEETRTSRARHSK SEQ ID No:36, TRSRKQTSHRLKNIPVHK SEQ ID No:37, SHQYLVIKLIPNASLIEK SEQ ID No:38, KLIPNASLIENCTKAELK SEQ ID No:39, AELGEYEKLLNSVLEPIK SEQ ID No:40, TAAQITAGIALHQSNLNK SEQ ID No:41 and PRYIATNGYLISNFDESK SEQ ID No:42.

20. The lipopeptide according to claim 9 wherein the lipid moiety is a lipoamino acid moiety.

21. The lipopeptide according to claim 20 wherein the lipoamino acid moiety selected from the group consisting of selected from the group consisting of Pam₂Cys, Pam₃Cys, Ste₂Cys, Lau₂Cys, Oct₂Cys, Pam₂Asp, Pam₃Asp, Ste₂Asp, Lau₂Asp, and Oct₂Asp.

22. The lipopeptide according to claim 21 wherein the lipid moiety is Pam₂Cys or Pam₃Cys.

23. The lipopeptide according to claim 9 wherein the lipid moiety is linked to the first region via a spacer.

24. The lipopeptide according to claim 21 wherein the spacer comprises arginine or serine dimers, trimers or teramers.

25. The lipopeptide according to claim 21 wherein the spacer comprises 6-aminohexanoic acid.

26. A method of generating an anti LHRH response in an animal, the method comprising administering to the animal the peptide according to claim 1.

27. The use of the peptide according to claim 1 in the preparation of a medicament to induce an anti LHRH response in an animal.

28. The peptide according to claim 2 wherein the first region consists of less than 20 amino acids.

29. The peptide according to claim 3 wherein the first region comprises 1 T helper cell epitope.

30. The lipopeptide according to claim 10 wherein the first region consists of less than 20 amino acids.

31. The lipopeptide according to claim 11 wherein the first region comprises 1 T helper cell epitope.

32. A method of generating an anti LHRH response in an animal, the method comprising administering to the animal the composition of claim 7.

33. A method of generating an anti LHRH response in an animal, the method comprising administering to the animal the lipopeptide of claim 9.

34. The use of the lipopeptide of claim 9 in the preparation of a medicament to induce an anti LHRH response in an animal.


SEQUENCE LISTING (57 sequences)

SEQ ID NO: 1 - protein, 10 aa, Unknown
  Mammalian LHRH (Luteinising hormone releasing hormone)
EHWSYGLRPG

SEQ ID NO: 2 - protein, 7 aa, Unknown
  amino acids 4 to 10 of mammalian LHRH
SYGLRPG

SEQ ID NO: 3 - protein, 17 aa, Unknown
  Mammalian T helper cell epitope
SSKTQTHTQQDRPPQPS

SEQ ID NO: 4 - protein, 17 aa, Unknown
  Mammalian T helper cell epitope
QPSTELEETRTSRARHS

SEQ ID NO: 5 - protein, 17 aa, Unknown
  Mammalian T helper cell epitope
RHSTTSAQRSTHYDPRT

SEQ ID NO: 6 - protein, 17 aa, Unknown
  Mammalian T helper cell epitope
PRTSDRPVSYTMNRTRS

SEQ ID NO: 7 - protein, 17 aa, Unknown
  Mammalian T helper cell epitope
TRSRKQTSHRLKNIPVH

SEQ ID NO: 8 - protein, 17 aa, Unknown
  Mammalian T helper cell epitope
SHQYLVIKLIPNASLIE

SEQ ID NO: 9 - protein, 17 aa, Unknown
  Mammalian T helper cell epitoper
IGTDNVHYKIMTRPSHQ

SEQ ID NO: 10 - protein, 17 aa, Unknown
  Mammalian T helper cell epitope
YKIMTRPSHQYLVIKLI

SEQ ID NO: 11 - protein, 17 aa, Unknown
  Mammalian T helper cell epitope
KLIPNASLIENCTKAEL

SEQ ID NO: 12 - protein, 17 aa, Unknown
  Mammalian T helper cell epitope
AELGEYEKLLNSVLEPI

SEQ ID NO: 13 - protein, 17 aa, Unknown
  Mammalian T helper cell epitope
KLLNSVLEPINQALTLM

SEQ ID NO: 14 - protein, 17 aa, Unknown
  Mammalian T helper cell epitope
EPINQALTLMTKNVKPL

SEQ ID NO: 15 - protein, 16 aa, Unknown
  Mammalian T helper cell epitope
FAGWLAGVALGVATAA

SEQ ID NO: 16 - protein, 17 aa, Unknown
  Mammalian T helper cell epitope
GVALGVATAAQITAGIA

SEQ ID NO: 17 - protein, 17 aa, Unknown
  Mammalian T helper cell epitope
TAAQITAGIALHQSNLN

SEQ ID NO: 18 - protein, 17 aa, Unknown
  Mammalian T helper cell epitope
GIALHQSNLNAQAIQSL

SEQ ID NO: 19 - protein, 17 aa, Unknown
  Mammalian T helper cell epitope
NLNAQAIQSLRTSLEQS

SEQ ID NO: 20 - protein, 17 aa, Unknown
  Mammalian T helper cell epitope
QSLRTSLEQSNKAIEEI

SEQ ID NO: 21 - protein, 17 aa, Unknown
  Mammalian T helper cell epitope
EQSNKAIEEIREATQET

SEQ ID NO: 22 - protein, 17 aa, Unknown
  Mammalian T helper cell epitope
TELLSIFGPSLRDPISA

SEQ ID NO: 23 - protein, 17 aa, Unknown
  Mammalian T helper cell epitope
PRYIATNGYLISNFDES

SEQ ID NO: 24 - protein, 17 aa, Unknown
  Mammalian T helper cell epitope
CIRGDTSSCARTLVSGT

SEQ ID NO: 25 - protein, 17 aa, Unknown
  Mammalian T helper cell epitope
DESSCVFVSESAICSQN

SEQ ID NO: 26 - protein, 17 aa, Unknown
  Mammalian T helper cell epitope
TSTIINQSPDKLLTFIA

SEQ ID NO: 27 - protein, 17 aa, Unknown
  Mammalian T helper cell epitope
SPDKLLTFIASDTCPLV

SEQ ID NO: 28 - protein, 16 aa, Unknown
  Mammalian T helper cell epitope
SGRRQRRFAGWLAGVA

SEQ ID NO: 29 - protein, 24 aa, Artificial
  Mammalian T helper cell epitope plus LHRH amino acids 4-10
QPSTELEETRTSRARHSSYGLRPG

SEQ ID NO: 30 - protein, 24 aa, Artificial
  Mammalian T helper cell epitope plus LHRH amino acids 4-10
TRSRKQTSHRLKNIPVHSYGLRPG

SEQ ID NO: 31 - protein, 24 aa, Artificial
  Mammalian T helper cell epitope plus LHRH amino acids 4-10
SHQYLVIKLIPNASLIESYGLRPG

SEQ ID NO: 32 - protein, 24 aa, Artificial
  Mammalian T helper cell epitope plus LHRH amino acids 4-10
KLIPNASLIENCTKAELSYGLRPG

SEQ ID NO: 33 - protein, 24 aa, Artificial
  Mammalian T helper cell epitope plus LHRH amino acids 4-10
AELGEYEKLLNSVLEPISYGLRPG

SEQ ID NO: 34 - protein, 24 aa, Artificial
  Mammalian T helper cell epitope plus LHRH amino acids 4-10
TAAQITAGIALHQSNLNSYGLRPG

SEQ ID NO: 35 - protein, 24 aa, Artificial
  Mammalian T helper cell epitope plus LHRH amino acids 4-10
PRYIATNGYLISNFDESSYGLRPG

SEQ ID NO: 36 - protein, 18 aa, Unknown
  Mammalian T helper cell epitope plus C-terminal lysine
QPSTELEETRTSRARHSK

SEQ ID NO: 37 - protein, 18 aa, Unknown
  Mammalian T helper cell epitope plus C-terminal lysine
TRSRKQTSHRLKNIPVHK

SEQ ID NO: 38 - protein, 18 aa, Unknown
  Mammalian T helper cell epitope plus C-terminal lysine
SHQYLVIKLIPNASLIEK

SEQ ID NO: 39 - protein, 18 aa, Unknown
  Mammalian T helper cell epitope plus C-terminal lysine
KLIPNASLIENCTKAELK

SEQ ID NO: 40 - protein, 18 aa, Unknown
  Mammalian T helper cell epitope plus C-terminal lysine
AELGEYEKLLNSVLEPIK

SEQ ID NO: 41 - protein, 18 aa, Unknown
  Mammalian T helper cell epitope plus C-terminal lysine
TAAQITAGIALHQSNLNK

SEQ ID NO: 42 - protein, 18 aa, Unknown
  Mammalian T helper cell epitope plus C-terminal lysine
PRYIATNGYLISNFDESK

SEQ ID NO: 43 - protein, 5 aa, Artificial
  amino acids 6 to 10 of mammalian LHRH
GLRPG

SEQ ID NO: 44 - protein, 12 aa, Artificial
  LHRH-related motif
ANDLAHWEPMSF

SEQ ID NO: 45 - protein, 12 aa, Artificial
  LHRH-related motif
MCELCHWPSASR

SEQ ID NO: 46 - protein, 12 aa, Artificial
  LHRH-related motif
HSPTFHGLPPGL

SEQ ID NO: 47 - protein, 12 aa, Artificial
  LHRH-related motif
AHLRPAPGDPWF

SEQ ID NO: 48 - protein, 12 aa, Artificial
  LHRH-related motif
MYTQQRHWSEGL

SEQ ID NO: 49 - protein, 12 aa, Artificial
  LHRH-related motif
TKIQEELAHWSP

SEQ ID NO: 50 - protein, 12 aa, Artificial
  LHRH-related motif
DSSSMLHWTYSL

SEQ ID NO: 51 - protein, 12 aa, Artificial
  LHRH-related motif
HWEYRVRTYPQT

SEQ ID NO: 52 - protein, 12 aa, Artificial
  LHRH-related motif
APSLLHWGPSFW

SEQ ID NO: 53 - protein, 12 aa, Artificial
  LHRH-related motif
GKLVWSDSFSTA

SEQ ID NO: 54 - protein, 10 aa, Artificial
  LHRH-related motif
QYEHWSHGLT

SEQ ID NO: 55 - protein, 12 aa, Artificial
  LHRH-related motif
GELDHWSGKPPH

SEQ ID NO: 56 - protein, 12 aa, Artificial
  LHRH-related motif
DAPHMRHWSWAL

SEQ ID NO: 57 - protein, 12 aa, Artificial
  LHRH-related motif
RPPLSELSHWHP